(19) United States
(12) Patent Application Publication
(10) Pub. No.: US 2016/0280641 A1
(43) Pub. Date: Sep. 29, 2016

(54) Method for refining 2-nito-4-methylsulfonyl benzoic acid and intermediate thereof

(71) Applicants: Yutian Shao, Hangzhou (CN); Guoping Cai, Hangzhou (CN); Jie Xiong, Hangzhou (CN); Bangchi Chen, Hangzhou (CN)

(72) Inventors: Yutian Shao, Hangzhou (CN); Guoping Cai, Hangzhou (CN); Jie Xiong, Hangzhou (CN); Bangchi Chen, Hangzhou (CN)

(21) Appl. No.: 15/034,526

(22) PCT Filed: Nov. 4, 2013
(86) PCT No.: PCT/CN2013/086482

§ 371 Date: May 4, 2016

(51) Int. Cl. C07C 315/06; C07C 317/44

(52) U.S. Cl. CPC C07C 315/06; C07C 317/44

(57) ABSTRACT

A method for refining a 2-nitro-4-methylsulfonyl benzoic acid and an intermediate thereof are provided, wherein the method includes steps of: using a crude 2-nitro-4-methylsulfonyl benzoic acid (I) as a raw material; using organic amine (II) as a base; synthesizing an intermediate, which is an amine salt (III), in an organic solvent; treating the amine salt (III) with an inorganic base in an aqueous phase to obtain an inorganic metal salt (IV) or an inorganic metal salt (IV′); and acidifying by adding an inorganic acid to obtain a fine 2-nitro-4-methylsulfonyl benzoic acid (I).

### CROSS REFERENCE OF RELATED APPLICATION

This is a U.S. National Stage under 35 U.S.C 371 of the International Application PCT/CN2013/086482, filed Nov. 4, 2013.

### BACKGROUND OF THE PRESENT INVENTION

1. Field of Invention

The present invention relates to a method for refining organics and an intermediate thereof, and more particularly to a method for refining 2-nitro-4-methylsulfonyl benzoic acid and an intermediate thereof.

2. Description of Related Arts

Mesotrione [

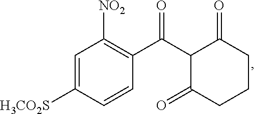

(2-(4-methanesulfonyl-2-nitrobenzoyl)-1, 3-cyclohexanedione)] is a trikeone herbicide with broad herbicidal spectrum, high activity, flexible application, strong mixability, safety for subsequent crop, and strong environmental compatibility. Mesotrione has high application prospect, especially for cornfield weeding where it displaces unique advantages. Mesotrione may be prepared with 2-nitro-4-methylsulfonyl benzoic acid as a raw material, according to the following processes (U.S. Pat. No. 4,695,673):

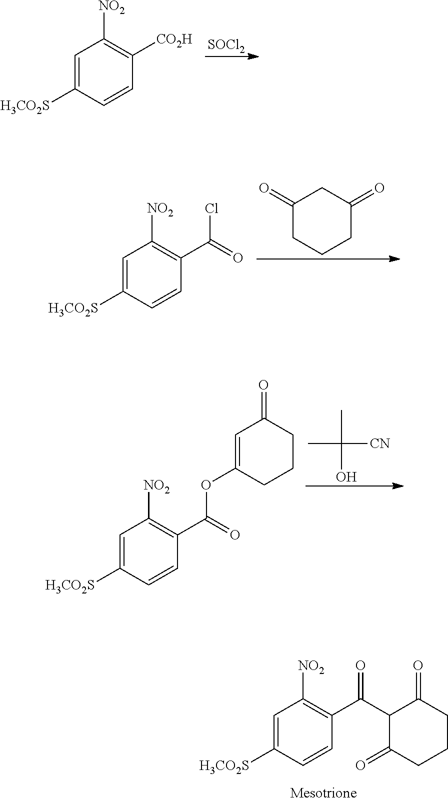

In 2001, K. Javdani et al. (WO02076934, CN1250525, and U.S. Pat. No. 7,285,678) pointed out that certain impurities in the raw material 2-nitro-4-methylsulfonyl benzoic acid will lead to less pure product, resulting in a positive Ames test result, which is not a feature of mesotrione, but a result derived from the impurities in 2-nitro-4-methylsulfonyl benzoic acid after the above processes. Thus, K. Javdani et al. invented a method for purifying 2-nitro-4-methylsulfonyl benzoic acid: firstly, dissolving crude 2-nitro-4-methylsulfonyl benzoic acid in water at pH 2-10, filtering out insoluble materials thereof; secondly, treating the filtrate with activated carbon, filtering out solid precipitate, and using sufficient amount of base to treat the filtrate in order to hydrolyze the undesired nitro and dinitro substituted impurities; and finally, adjusting pH at 95° C. until acidic, and cooling for precipitating fine 2-nitro-4-methylsulfonyl benzoic acid. Unfortunately, in 2005, A. H. Benke et al. (CN100378071 and UA82722) found that although the above refinement processes may remove a portion of the impurities in the 2-nitro-4-methylsulfonyl benzoic acid, the final product mesotrione prepared with the 2-nitro-4-methylsulfonyl benzoic acid obtained above still comprises undesirable level of impurities.

### SUMMARY OF THE PRESENT INVENTION

An objective of the present invention is to overcome the shortcomings of the current technologies, and provide a method for refining a 2-nitro-4-methylsulfonyl benzoic acid and an intermediate thereof.

A method for refining a 2-nitro-4-methylsulfonyl benzoic acid comprises steps of: using a crude 2-nitro-4-methylsulfonyl benzoic acid (I) as a raw material; using organic amine (II) as a base; synthesizing an intermediate, which is an amine salt (III), in an organic solvent; treating the amine salt (III) with an inorganic base in an aqueous phase to obtain an inorganic metal salt (IV) or an inorganic metal salt (IV′); and acidifying by adding an inorganic acid to obtain a fine 2-nitro-4-methylsulfonyl benzoic acid (I); whose chemical reactions are as follows:

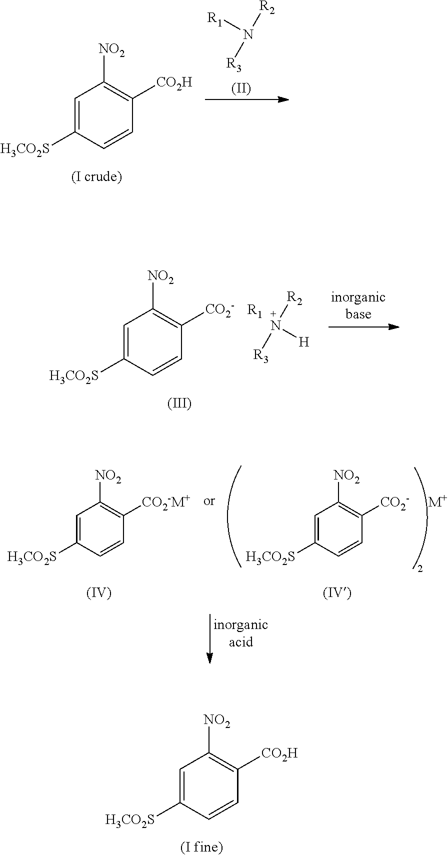

wherein R₁, R₂ and R₃ are hydrogen, alkyl groups with 1-6 carbon atoms, or aryl groups, wherein the alkyl groups comprises branched chains, linear chains and cycloalkyl; R₁, R₂, and R₃ groups are same or different; M is an alkali metal, and M′ is an alkaline earth metal.

Another method for refining a 2-nitro-4-methylsulfonyl benzoic acid comprises steps of: using a crude 2-nitro-4-methylsulfonyl benzoic acid (I) as a raw material; using organic amine (II) as a base; synthesizing an intermediate, which is an amine salt (III), in an organic solvent; and acidifying by adding an inorganic acid to obtain a fine 2-nitro-4-methylsulfonyl benzoic acid; whose chemical reactions are as follows:

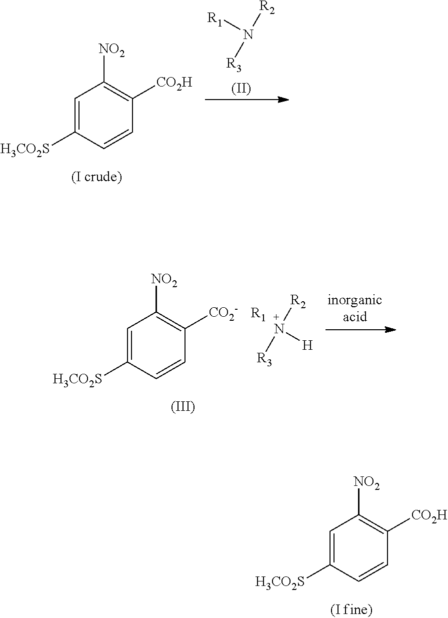

wherein R₁, R₂ and R₃ are hydrogen, alkyl groups with 1-6 carbon atoms, or aryl groups, wherein the alkyl groups comprises branched chains, linear chains and cycloalkyl; R₁, R₂, and R₃ groups are same or different.

The organic amine (II) is selected from a group consisting of n-propylamine, n-hexylamine, dicyclohexylamine, and triethylamine, or a mixture of the two or more of them, wherein an amount of the organic amine (II) added is 0.25-5 times of an amount of the crude 2-nitro-4-methylsulfonyl benzoic acid (I).

The organic solvent is selected from a group consisting of acetone, toluene, chloroform, and methanol, wherein a volume ratio of the organic solvent to the crude 2-nitro-4-methylsulfonyl benzoic acid (I) is 2-20 mL/g.

The inorganic base is selected from a group consisting of an alkali metal hydroxide, an alkaline earth metal hydroxide, an alkali metal carbonate, and alkaline earth metal carbonate, an alkali metal bicarbonate, and an alkaline earth metal bicarbonate.

The inorganic acid is a hydrochloric acid or a sulfuric acid.

An intermediate is a 2-nitro-4-methylsulfonyl benzoic acid amine salt, and a molecular structure thereof is:

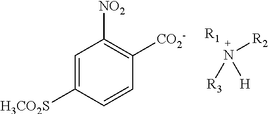

wherein R₁, R₂ and R₃ are hydrogen, alkyl groups with 1-6 carbon atoms, or aryl groups, wherein the alkyl groups comprises branched chains, linear chains and cycloalkyl; R₁, R₂, and R₃ groups are same or different.

The synthesizing and refining processes described in the present invention is simple in operation, safe and reliable, highly efficient for removing undesirable impurities, which affords product with high purity specification and is advantageous for industrial production.

### DETAILED DESCRIPTION OF THE PREFERRED EMBODIMENT

A method for refining a 2-nitro-4-methylsulfonyl benzoic acid comprises steps of: using a crude 2-nitro-4-methylsulfonyl benzoic acid (I) as a raw material; using organic amine (II) as a base; synthesizing an intermediate, which is an amine salt (III), in an organic solvent; treating the amine salt (III) with an inorganic base in an aqueous phase to obtain an inorganic metal salt (IV) or an inorganic metal salt (IV′); and acidifying by adding an inorganic acid to obtain a fine 2-nitro-4-methylsulfonyl benzoic acid (I); whose chemical reactions are as follows:

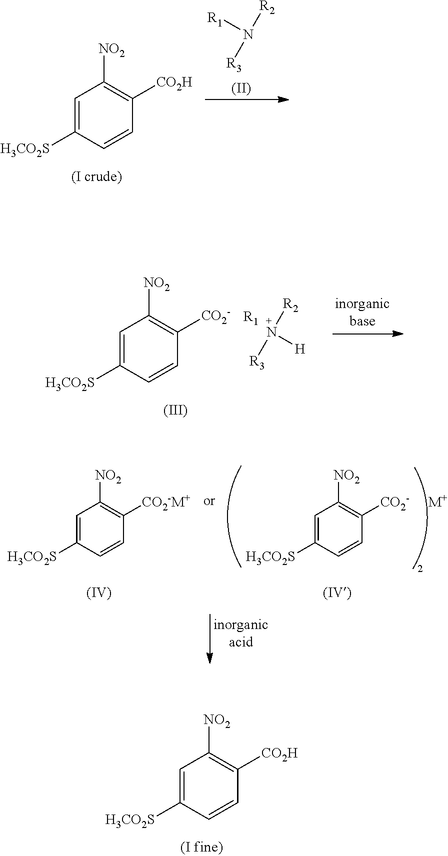

wherein R₁, R₂ and R₃ are hydrogen, alkyl groups with 1-6 carbon atoms, or aryl groups, wherein the alkyl groups comprises branched chains, linear chains and cycloalkyl;

R₁, R₂, and R₃ groups are same or different; M is an alkali metal, and M′ is an alkaline earth metal.

Another method for refining 2-nitro-4-methylsulfonyl benzoic acid comprises steps of: using a crude 2-nitro-4-methlsulfonyl benzoic acid (I) as a raw material; using organic amine (II) as a base; synthesizing an intermediate, which is an amine salt (III), in an organic solvent; and acidifying by adding an inorganic acid to obtain a fine 2-nitro-4-methylsulfonyl benzoic acid; whose chemical reactions are as follows:

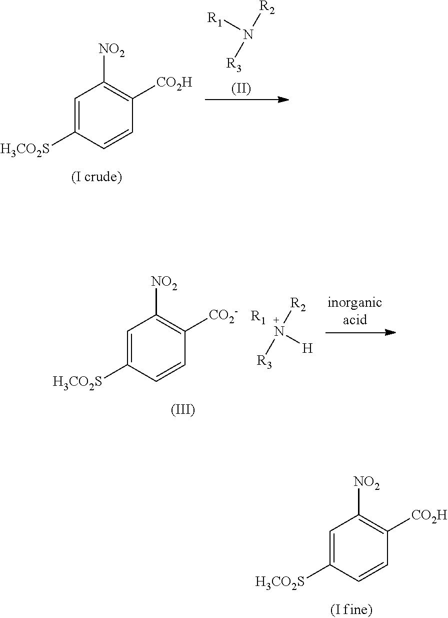

wherein R₁, R₂ and R₃ are hydrogen, alkyl groups with 1-6 carbon atoms, or aryl groups, wherein the alkyl groups comprises branched chains, linear chains and cycloalkyl; R₁, R₂, and R₃ groups are same or different.

The organic amine (II) is selected from a group consisting of n-propylamine, n-hexylamine, dicyclohexylamine, and triethylamine, wherein an amount of the organic amine (II) added is 0.25-5 times of an amount of the crude 2-nitro-4-methylsulfonyl benzoic acid (I).

The organic solvent is selected from a group consisting of acetone, toluene, chloroform, and methanol, wherein a volume ratio of the organic solvent to the crude 2-nitro-4-methylsulfonyl benzoic acid (I) is 2-20 mL/g.

The inorganic base is selected from a group consisting of an alkali metal hydroxide, an alkaline earth metal hydroxide, an alkali metal carbonate, an alkaline earth metal carbonate, an alkali metal bicarbonate, and an alkaline earth metal bicarbonate.

The inorganic acid is a hydrochloric acid or a sulfuric acid.

An intermediate is a 2-nitro-4-methylsulfonyl benzoic acid amine salt, and a formula thereof is:

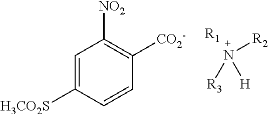

wherein R₁, R₂ and R₃ are hydrogen, alkyl groups with 1-6 carbon atoms, or aryl groups, wherein the alkyl groups comprises branched chains, linear chains and cycloalkyl; R₁, R₂, and R₃ groups are same or different.

Features of the present invention are further illustrated by the following preferred embodiments, which are exemplary only and not intended to be limiting.

### EXAMPLE 1

Adding 52.7 g crude 2-nitro-4-methylsulfonyl benzoic acid (85.0%) and 400.0 ml acetone into a 500 ml three-neck reaction flask, drop adding 36.0 g dicyclohexylamine with stirring; heating and stirring for 2.5 h, cooling, vacuum-filtering, washing with acetone, and drying to obtain 77.2 g 2-nitro-4-methylsulfonyl benzoic acid dicyclohexyl amine salt, wherein the nuclear magnetic resonance spectral data thereof are:

¹HNMR(500 MHz, D₂O): δ 8.620-8.622(d, 1 H), 8.24-8.26(dd, 1 H), 7.71-7.73(d, 1 H), 3.28(s, 3 H), 3.17-3.19(dd, 2 H), 1.97-1.98(d, 4 H), 1.75-1.77(t, 4 H), 1.59-1.62(d, 2 H), 1.22-1.27(m, 8 H), 1.10-1.11(d, 2 H);

Adding 76.0 g the 2-nitro-4-methylsulfonyl benzoic acid dicyclohexylamine salt obtained above into a 500 ml three-neck round bottom flask and mixing with 350.0 ml water, adjusting pH to 13 with potassium hydroxide, separating layers by toluene, and adjusting pH of the aqueous phase to 3 with hydrochloric acid; then filtering and washing the filter cake with water, and drying to obtain 44.1 g (with a yield of 98.45% and purity of 99.5%) fine 2-nitro-4-methylsulfonyl benzoic acid, wherein the nuclear magnetic resonance spectral data thereof are:

¹HNMR(500 MHz, CDCl₃): δ 14.40(s, 1 H), 5.52-5.53(d, 1 H), 8.32-8.33(dd, 1 H), 8.10-8.12(d, 1 H), 3.39(s, 3 H).

### EXAMPLE 2

Adding 100.0 g crude 2-nitro-4-methylsulfonyl benzoic acid (88.0%) and 800.0 ml methanol into a 1000 ml three-neck reaction flask, drop adding 43.6 g n-hexylamine with stirring; heating and stirring for 2.5 h, cooling, vacuum-filtering, washing with methanol, and drying to obtain 123.03 g 2-nitro-4-methylsulfonyl benzoic acid n-hexylamine salt, wherein the nuclear magnetic resonance spectral data thereof are:

¹HNMR(500 MHz, D₂O): δ 8.611-8.610 (d, 1 H), 8.23-8.25(dd, 1 H), 7.70-7.72(d, 1 H), 3.27(s, 3H), 2.90-2.93(t, 2 H), 1.56-1.59(t, 2 H), 1.21-1.31(m, 6 H), 0.78-0.80(t, 3 H);

Adding the 123.03g 2-nitro-4-methylsulfonyl benzoic acid n-hexylamine salt and 700.0 ml water into a 1000 ml three-neck round bottom flask, adjusting pH to 13 with potassium hydroxide, separating layers by toluene, and adjusting pH of the aqueous phase to 3 with hydrochloric acid; then filtering and washing the filter cake with water, and drying to obtain 86.2 g (with a yield of 98% and purity of 99.5%) fine 2-nitro-4-methylsulfonyl benzoic acid.

### EXAMPLE 3

Adding 100.0 g crude 2-nitro-4-methylsulfonyl benzoic acid (88.0%), 100.0 ml acetone and 100 ml methanol into a 1000 ml three-neck reaction flask, drop adding 25.5 g n-propylamine with stirring; heating and stirring for 2.0 h, cooling, vacuum-filtering, washing with acetone, and drying to obtain 108.8 g 2-nitro-4-methylsulfonyl benzoic acid n-propylamine salt, wherein the nuclear magnetic resonance spectral data thereof are:

¹-HNMR(500 MHz, D₂O): δ 8.608-8.611 (d, 1 H), 8.23-8.25(dd, 1 H), 7.70-7.72(d, 1 H), 3.27(s, 3 H), 2.87-2.90(t, 2 H), 1.58-1.62(m, 2 H), 0.88-0.91(t, 3 H);

Adding the 108.8 g 2-nitro-4-methylsulfonyl benzoic acid n-propylamine salt and 350.0 ml 15% potassium carbonate into a 500 ml three-neck round bottom flask, heating and stirring for dissolving, extracting with toluene, and separating the aqueous phase and adjusting to acidic; then filtering and washing the filter cake with water, and drying to obtain 84.9 g (with a yield of 96.5% and purity of 99.0%) fine 2-nitro-4-methylsulfonyl benzoic acid.

### EXAMPLE 4

Adding 20 g the 2-nitro-4-methylsulfonyl benzoic acid dicyclohexyl amine salt obtained in the example 1 into a 250 ml three-neck round bottom flask, then adding 75.0 ml water; adjusting pH of the aqueous phase to 3 with hydrochloric acid at room temperature; then filtering, washing and drying to obtain 10.91 g (with a yield of 95.0% and purity of 99.0%) fine 2-nitro-4-methylsulfonyl benzoic acid.

### EXAMPLE 5

Adding 25.0 g crude 2-nitro-4-methylsulfonyl benzoic acid (83.3%) and 500.0 ml toluene into a 1000 ml three-neck reaction flask, drop adding 500 g triethylamine with stirring; heating and stirring for 4.0 h, cooling, vacuum-filtering, washing with acetone, and drying to obtain 27.0 g 2-nitro-4-methylsulfonyl benzoic acid triethylamine salt, wherein the nuclear magnetic resonance spectral data thereof are:

¹HNMR(500 MHz, D₂O): δ 8.601-8.604 (d, 1 H), 8.23-8.25(dd, 1 H), 7.71-7.72(d, 1 H), 3.27(s, 3H), 3.10-3.27(q, 6 H), 1.19-1.22(t, 9 H);

Adding the 27.0 g 2-nitro-4-methylsulfonyl benzoic acid triethylamine salt and 270.0 ml water into a 500 ml three-neck round bottom flask, adjusting pH to 12-13 with calcium hydroxide, separating layers by toluene, and adjusting pH of the aqueous phase to 3 with sulphuric acid; then filtering and washing the filter cake with water, and drying to obtain 18.7 g (with a yield of 87.9% and purity of 99.5%) fine 2-nitro-4-methylsulfonyl benzoic acid.

### EXAMPLE 6

Adding 50.0 g crude 2-nitro-4-methylsulfonyl benzoic acid (88.0%) and 1000.0 ml chloroform into a 1000 ml three-neck reaction flask, drop adding 43.6 g n-hexylamine with stirring; heating and stirring for 2.5 h, cooling, vacuum-filtering, washing with chloroform, and drying to obtain 61.5 g 2-nitro-4-methylsulfonyl benzoic acid n-hexylamine salt, wherein the nuclear magnetic resonance spectral data thereof are:

¹HNMR(500 MHz, D₂O): δ 8.611-8.610 (d, 1 H), 8.23-8.25(dd, 1 H), 7.70-7.72(d, 1 H), 3.27(s, 3 H), 2.90-2.93(t, 2 H), 1.56-1.59(t, 2 H), 1.21-1.31(m, 6 H), 0.78-0.80(t, 3 H);

Adding the 61.5 g 2-nitro-4-methylsulfonyl benzoic acid n-hexylamine salt and 700.0 ml water into a 1000 ml three-neck round bottom flask, adjusting pH to 13 with barium hydroxide, separating layers by toluene and adjusting pH of the aqueous phase to 3 with sulfuric acid; then filtering and washing the filter cake with water, and drying to obtain 43.1 g (with a yield of 98% and purity of 99.5%) fine 2-nitro-4-methylsulfonyl benzoic acid.

### EXAMPLE 7

Adding 52.7 g crude 2-nitro-4-methylsulfonyl benzoic acid (85.0%), 400.0 ml acetone and 250 ml toluene into a 500 ml three-neck reaction flask, drop adding 36.0 g dicyclohexyl amine with stirring; heating and stirring for 2.5 h, cooling, vacuum-filtering, washing with acetone, and drying to obtain 76.0 g 2-nitro-4-methylsulfonyl benzoic acid dicyclohexyl amine salt, wherein the nuclear magnetic resonance spectral data thereof are:

¹HNMR(500 MHz, D₂O): δ 8.620-8.622(d, 1 H), 8.24-8.26(dd, 1 H), 7.71-7.73(d, 1 H), 3.28(s, 3 H), 3.17-3.19(dd, 2 H), 1.97-1.98(d, 4H), 1.75-1.77(t, 4 H), 1.59-1.62(d, 2 H), 1.22-1.27(m, 8 H), 1.10-1.11(d, 2 H);

Adding the 76.0g 2-nitro-4-methylsulfonyl benzoic acid dicyclohexyl amine salt into a 500 ml three-neck round bottom flask and mixing with 350.0 ml water, adjusting pH to 12 with sodium hydrogen carbonate, separating layers by toluene, and adjusting pH of the aqueous phase to 3 with hydrochloric acid; then filtering and washing the filter cake with water, and drying to obtain 44.1g (with a yield of 98.45% and purity of 99.5%) fine 2-nitro-4-methylsulfonyl benzoic acid, wherein the nuclear magnetic resonance spectral data thereof are:

¹HNMR(500 MHz, CDCl): δ 14.40(s, 1 H), 5.52-5.53(d, 1 H), 8.32-8.33(dd, 1 H), 8.10-8.12(d, 1 H), 3.39(s, 3 H).

The fine 2-nitro-4-methylsulfonyl benzoic acids obtained by different processes are transformed into mesotrione products, and activities thereof are tested in Ames tests, wherein Ames responses are negative.

1-7. (canceled)

8. A method for refining a 2-nitro-4-methylsulfonyl benzoic acid, comprising steps of: using a crude 2-nitro-4-methylsulfonyl benzoic acid (I) as a raw material; using organic amine (II) as a base; synthesizing an intermediate, which is an amine salt (III), in an organic solvent; treating the amine salt (III) with an inorganic base in an aqueous phase for obtaining an inorganic metal salt (IV) or an inorganic metal salt (IV′); and acidifying by adding an inorganic acid to obtain a fine 2-nitro-4-methylsulfonyl benzoic acid (I); whose chemical reactions are as follows: 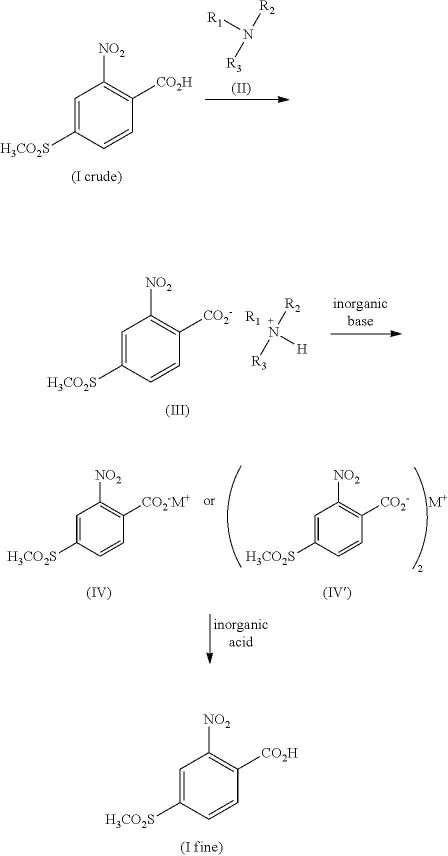
wherein R₁, R₂ and R₃ are hydrogen, alkyl groups with 1-6 carbon atoms, or aryl groups, wherein the alkyl groups comprises branched chains, linear chains and cycloalkyl;
R₁, R₂, and R₃ groups are same or different; M is an alkali metal, and M′ is an alkaline earth metal.

9. A method for refining a 2-nitro-4-methylsulfonyl benzoic acid, comprising steps of: using a crude 2-nitro-4-methylsulfonyl benzoic acid (I) as a raw material; using organic amine (II) as a base; synthesizing an intermediate, which is an amine salt (III), in an organic solvent; and acidifying by adding an inorganic acid to obtain a fine 2-nitro-4-methylsulfonyl benzoic acid; whose chemical reactions are as follows: 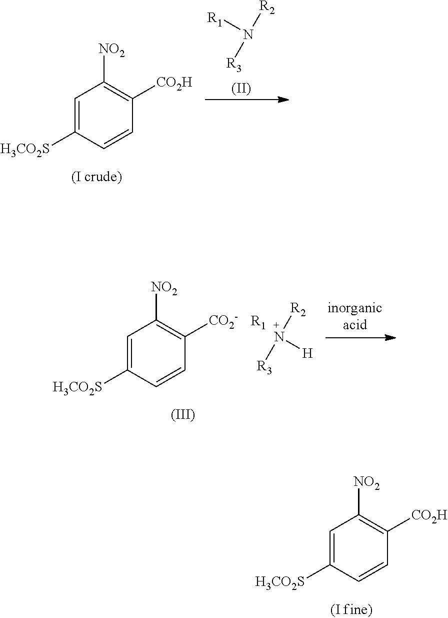
wherein R₁, R₂ and R₃ are hydrogen, alkyl groups with 1-6 carbon atoms, or aryl groups, wherein the alkyl groups comprises branched chains, linear chains and cycloalkyl;
R₁, R₂, and R₃ groups are same or different.

10. The method according to claim 8, wherein the organic amine (II) is selected from a group consisting of n-propylamine, n-hexylamine, dicyclo hexylamine, and triethylamine, wherein an amount of the organic amine (II) added is 0.25-5 times of an amount of the crude 2-nitro-4-methylsulfonyl benzoic acid (I).

11. The method according to claim 9, wherein the organic amine (II) is selected from a group consisting of n-propylamine, n-hexylamine, dicyclo hexylamine, and triethylamine, wherein an amount of the organic amine (II) added is 0.25-5 times of an amount of the crude 2-nitro-4-methylsulfonyl benzoic acid (I).

12. The method according to claim 8, wherein the organic solvent is selected from a group consisting of acetone, toluene, chloroform, and methanol, wherein a volume ratio of the organic solvent to the crude 2-nitro-4-methylsulfonyl benzoic acid (I) is 2-20 mL/g.

13. The method according to claim 9, wherein the organic solvent is selected from a group consisting of acetone, toluene, chloroform, and methanol, wherein a volume ratio of the organic solvent to the crude 2-nitro-4-methylsulfonyl benzoic acid (I) is 2-20 mL/g.

14. The method according to claim 8, wherein the inorganic base is selected from a group consisting of an alkali metal hydroxide, an alkaline earth metal hydroxide, an alkali metal carbonate, an alkaline earth metal carbonate, an alkali metal bicarbonate, and an alkaline earth metal bicarbonate.

15. The method according to claim 8, wherein the inorganic acid is hydrochloric acid or sulfuric acid.

16. The method according to claim 9, wherein the inorganic acid is hydrochloric acid or sulfuric acid.

17. An intermediate compound, wherein an intermediate thereof is a 2-nitro-4-methylsulfonyl benzoic acid amine salt, and a molecular structure thereof is: 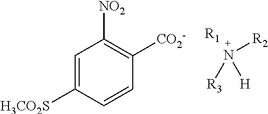
wherein R₁, R₂ and R₃ are hydrogen, alkyl groups with 1-6 carbon atoms, or aryl groups, wherein the alkyl groups comprises branched chains, linear chains and cycloalkyl;
R₁, R₂, and R₃ groups are same or different, wherein at most two of R₁, R₂, and R₃ are hydrogen.
